(19) United States
(12) Patent Application Publication
(10) Pub. No.: US 2025/0216581 A1
(43) Pub. Date: Jul. 3, 2025

(54) SYSTEMS AND METHODS FOR MODELING AND SIMULATING REACTIVE TRANSPORT IN POROUS MEDIA

(71) Applicant: BP Corporation North America Inc., Houston, TX (US)

(72) Inventors: Jeremy Todd First, Houston, TX (US); Yuliana Maria Zapata Arboleda, Katy, TX (US); Glen Leroy Gettemy, Katy, TX (US); Jonathan Hodgkinson, Huntsville, TX (US)

(73) Assignee: BP Corporation North America Inc., Houston, TX (US)

(21) Appl. No.: 19/002,290

(22) Filed: Dec. 26, 2024

Related U.S. Application Data
(60) Provisional application No. 63/615,559, filed on Dec. 28, 2023.

(51) Int. Cl. G01V 20/00

(52) U.S. Cl. CPC G01V 20/00

(57) ABSTRACT

A method for modeling reactive transport within a subterranean formation includes defining a chemical system for a digital image of rock from the subterranean formation, segmenting the digital image into a plurality of voxels including a plurality of solid voxels, a plurality of fluid voxels, and a plurality of interface voxels, simulating a concentration change over time for the plurality of interface voxels, and determining an updated concentration for each of the plurality of interface voxels.

### CROSS-REFERENCE TO RELATED APPLICATIONS

This application claims benefit of U.S. provisional patent application Ser. No. 63/615,559 filed Dec. 28, 2023, and entitled “Reactive Transport Simulation in Porous Media,” which is hereby incorporated herein by reference in its entirety for all purposes.

### STATEMENT REGARDING FEDERALLY SPONSORED RESEARCH OR DEVELOPMENT

Not applicable.

### BACKGROUND

In various applications, it may be desired to understand how fluid flows through and/or within a porous media. Such estimates may be dependent (at least partially) on the specific properties of the porous media and the fluid(s) flowing therein. As an example, in hydrocarbon exploration and production, the porous media in question may comprise an earthen subterranean formation (or more simply “subterranean formations” or “formations”). Obtaining accurate estimates of petrophysical properties of a subterranean formation may thus be important for characterizing the concentration of fluids and/or minerals within the formation, and modeling changes to the concentration of the fluids and/or minerals over time. Traditionally, samples are obtained from the formation (e.g., in the form of core samples or drilling cuttings) and subjected to laboratory testing whereby petrophysical properties such as permeability, porosity, formation factor, elastic moduli, and the like of the sample may be determined. Once determined, these properties can then be used to form predictions regarding the behavior of the subterranean formation which may inform a strategy for configuring well systems which may be utilized for a variety of purposes including, for example, extracting hydrocarbons or other resources from the formation, carbon capture and storage within the formation, and other applications. As an example, the determined properties may be utilized in determining the location of a planned wellbore for producing hydrocarbons from the subterranean formation and/or the configuration of the planned wellbore.

### BRIEF SUMMARY OF THE DISCLOSURE

An embodiment of a computer-implemented method for modeling reactive transport within a subterranean formation comprises (a) receiving a digital image of rock from the subterranean formation, (b) defining a chemical system for the digital image, (c) segmenting the digital image into a plurality of voxels including a plurality of solid voxels each associated with a solid phase mineral of the subterranean formation, a plurality of fluid voxels each associated with a fluid of the subterranean formation, and a plurality of interface voxels each associated with both the solid phase mineral and the fluid of the subterranean formation, (d) simulating a concentration change over time of the solid phase mineral of each of the plurality of interface voxels due to simulated chemical activity in the digital image, (e) determining an updated concentration of the solid phase mineral for each of the plurality of interface voxels of the digital image based on the simulated concentration change. In some embodiments, the method comprises (f) converting at least one of the plurality of interface voxels into either a solid voxel or a fluid voxel based on the updated concentration of the solid phase mineral for each of the plurality of interface voxels of the digital image. In certain embodiments, the method comprises (f) converting at least one of the plurality of solid voxels into an interface voxel in response to the updated concentration of the solid phase mineral of at least one of the plurality of interface voxels equaling a corresponding concentration of the solid phase mineral at a full density or molar volume of the solid phase mineral, wherein the concentration of the solid phase mineral corresponds to the number of mols of the solid phase mineral present in the interface voxel. In some embodiments, (f) comprises updating a geometry of an interface of the digital image defined by the spatial configuration of the plurality of interface voxels. In some embodiments, the at least one of the plurality of solid voxels converted into the interface voxel is located adjacent the at least one of the plurality of interface voxels having the updated concentration of the solid phase mineral equaling the density of the solid phase mineral. In certain embodiments, the method comprises (g) converting at least one of the plurality of fluid voxels into an interface voxel in response to the updated concentration of the solid phase mineral of at least one of the plurality of interface voxels equaling zero. In certain embodiments, the at least one of the plurality of fluid voxels converted into the interface voxel is located adjacent the at least one of the plurality of interface voxels having the updated concentration of the solid phase mineral equal to zero. In some embodiments, the method comprises (g) converting at least one of the plurality of interface voxels into either a solid voxel or a fluid voxel based on the updated concentration of the solid phase mineral for each of the plurality of interface voxels of the digital image. In some embodiments, (d) comprises (d1) independently performing a geochemical calculation between the solid phase mineral and the fluid for each of the plurality of interface voxels, (d2) simulating the concentration change over time of the solid phase mineral of each of the plurality of interface voxels based on a simulated change in a concentration of the fluid of the interface voxel and a predefined stochiometric relationship between the solid phase mineral and a solute component of the fluid, and (d3) determining a change in temperature associated with the concentration change over time of the solid phase mineral.

An embodiment of a system for modeling reactive transport within a subterranean formation comprises a processor, and a memory coupled to the processor, wherein machine-readable instructions are stored in the memory, and wherein the machine-readable instructions, when executed on the processor, configure the processor to (a) receiving a digital image of rock from the subterranean formation, (b) defining a chemical system for the digital image, (c) segmenting the digital image into a plurality of voxels including a plurality of solid voxels each having a defined solid voxel composition including a solid phase mineral of the subterranean formation, a plurality of fluid voxels each having a defined fluid voxel composition including one or more solute components of a fluid of the subterranean formation, and a plurality of interface voxels each having a defined interface voxel composition including both a solid phase mineral and one or more solute components, (d) simulating a concentration change over time of the solid phase mineral of each of the plurality of interface voxels due to simulated transport and chemical activity in the digital image, and (e) determining an updated concentration of the solid phase mineral for each of the plurality of interface voxels of the digital image based on the simulated concentration change.

An embodiment of a system for modeling reactive transport within a subterranean formation comprises a processor, and a memory coupled to the processor, wherein machine-readable instructions are stored in the memory, and wherein the machine-readable instructions, when executed on the processor, configure the processor to (a) receiving a digital image of rock from the subterranean formation, (b) defining a chemical system for the digital image, (c) segmenting the digital image into a plurality of voxels including a plurality of solid voxels each associated with a solid phase mineral of the subterranean formation, a plurality of fluid voxels each associated with a fluid of the subterranean formation, and a plurality of interface voxels each associated with both the solid phase mineral and the fluid of the subterranean formation, (d) simulating a concentration change over time of the solid phase mineral of each of the plurality of interface voxels due to simulated chemical activity in the digital image, (e) determining an updated concentration of the solid phase mineral for each of the plurality of interface voxels of the digital image based on the simulated concentration change, and (f) converting at least one of the plurality of solid voxels into an interface voxel in response to the updated concentration of the solid phase mineral of at least one of the plurality of interface voxels equaling a corresponding concentration of the solid phase mineral at a full density or molar volume of the solid phase mineral, wherein the concentration of the solid phase mineral corresponds to the number of mols of the solid phase mineral present in the interface voxel. In certain embodiments, the digital image of rock is a three-dimensional (3D) image.

An embodiment of a computer-implemented method for modeling reactive transport within a subterranean formation comprises (a) receiving a digital image of rock from the subterranean formation, (b) defining a chemical system for the digital image, (c) segmenting the digital image into a plurality of voxels at least some of which are associated with a solid phase mineral of the subterranean formation and at least some of which are associated with a fluid of the subterranean formation, (d) dividing the digital image into a plurality of chunks, each chunk comprising a separate portion of the plurality of voxels of the digital image, (e) assigning the plurality of chunks to a corresponding plurality of ranks of a message passing interface (MPI) program such that each rank is associated with a unique chunk of the digital image, (f) invoking, for each of the plurality of MPI ranks, a plurality of separate instances of a geochemical solver to simulate, in parallel, a concentration change over time of at least one of the solid phase mineral and the fluid of the digital image due to simulated transport and chemical activity in the digital image, and (g) determining an updated concentration of the at least one of the solid phase mineral and the fluid of the digital image based on the simulated concentration change. In certain embodiments, the method comprises (h) segmenting the digital image is segmented into a plurality of voxels which are divided between the plurality of chunks of the digital image whereby each chunk is associated with multiple distinct voxels of the plurality of voxels. In some embodiments, the plurality of voxels includes a plurality of solid voxels each associated with a solid phase mineral of the subterranean formation, a plurality of fluid voxels each associated with one or more fluids of the subterranean formation, and a plurality of interface voxels each associated with both a solid phase mineral of the subterranean formation and a fluid of the subterranean formation. In some embodiments, the method comprises (h) assigning a unique computer node of a computer architecture to each of the plurality of MPI ranks, wherein (f) comprises executing at least one of an advection-diffusion solver and the geochemical solver on the computer nodes assigned to the plurality of MPI ranks. In certain embodiments, each of the plurality of geochemical solver instances is assigned to a unique central processing unit (CPU) core of a computer architecture. In certain embodiments, the method comprises (h) assigning a unique set of central processing unit (CPU) cores of one or more computer nodes of a computer architecture to each of the plurality of MPI ranks, wherein (f) comprises executing at least one of an advection-diffusion solver and the geochemical solver on the CPU cores assigned to the plurality of MPI ranks. In some embodiments, (f) comprises executing a separate instance of the at least one of the advection-diffusion solver and the geochemical solver for each of the CPU cores assigned to the plurality of MPI ranks. In some embodiments, the method comprises (h) assigning a unique socket of one or more computer nodes of a computer architecture to each of the plurality of MPI ranks, wherein (f) comprises executing at least one of an advection-diffusion solver and the geochemical solver on the sockets assigned to the plurality of MPI ranks. In certain embodiments, the method comprises (h) communicating between the plurality of MPI ranks information pertaining to the digital image across an internode interconnect connected between a plurality of computer nodes of a computer architecture assigned to the plurality of MPI ranks. In certain embodiments, the method comprises (h) communicating between the plurality of MPI ranks information pertaining to the digital image across an intranode interconnect connected between a plurality of sockets of a computer node assigned to the plurality of MPI ranks.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIG. 1 is a block diagram of a method for modeling reactive transport within a subterranean formation according to some embodiments;

FIG. 2 is a schematic view of exemplary onshore and offshore sources of rock samples and fluid sampling for analysis by embodiments of testing systems and methods according to some embodiments;

FIG. 3 is a schematic view of an embodiment of a testing system for analyzing rock samples according to some embodiments;

FIG. 4 is a schematic representation of two-dimensional (2D) slices and three-dimensional (3D) views of a digital image of a rock sample according to some embodiments, including a tomograph, a segmented image, and a mineral-mapped image according to some embodiments;

FIG. 5 is a schematic diagram of the voxel classification in the 3D image of a rock sample according to some embodiments;

FIG. 6 is a schematic diagram of the streaming and collision steps of the lattice Boltzmann method, as well as different 3D lattice models according to some embodiments;

FIG. 7 is a schematic representation of an application of a modified lattice-Boltzmann solution for modeling pore-scale reactive transport according to some embodiments;

FIG. 8 is block diagram of a computer system according to some embodiments

FIG. 9 is a schematic depiction of a 3D digital image showing the parallelism scheme for transport and reaction calculations of an embodiment described herein.

FIG. 10 is a schematic depiction of an example computer architecture that may be used to perform the parallelism scheme depicted in FIG. 9.

FIG. 11 is a block diagram of a parallelism scheme for modeling reactive transport within a subterranean formation according to some embodiments.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis.

As previously described, an accurate understanding of material flow (e.g., fluid flow) within a porous media may be dependent upon specific parameters and characteristics of the porous media and materials contained therein and forming the porous media. When the porous media comprises a subterranean formation (e.g., such as in the context of hydrocarbon exploration and production), the properties and behavior of a subterranean formation may be directly measured via samples obtained from the formation itself (e.g., in the form of core samples, drill cuttings). However, due to the cost and time required to directly measure petrophysical properties of formation samples (also referred to herein as “rock samples”), the technique of “direct numerical simulation” can be applied to efficiently predict or estimate physical properties of the subterranean formation, such as porosity, absolute permeability, relative permeability, formation factor, elastic moduli, and the like of rock samples, including samples from difficult rock types such as tight gas sands or carbonates. According to this approach, a three-dimensional (3D) tomographic image of the rock sample is obtained, for example, by way of a computer tomographic (CT) scan. The 3D image is segmented into a plurality of distinct volume elements or “voxels” (e.g., by “thresholding” their brightness values or by another approach) to distinguish rock matrix from void space. Direct numerical simulation of fluid flow or other physical behavior such as elasticity or electrical conductivity is then performed, from which porosity, permeability, elastic properties, electrical properties, and the like can be derived. A variety of numerical methods may be applied to solve or approximate the physical equations simulating the appropriate behavior of the rock sample. These methods include, for example, lattice-Boltzmann (LB), finite element, finite difference, finite volume, numerical methods and the like.

Ultimately, as previously described, it is desirable to model the movement of fluid within a subterranean formation. Accurate modeling of such behavior may be relevant for hydrocarbon production operations (e.g., determining well placement and/or configuration), and also for other considerations such as contaminant transport and rock diagenesis. The movement of fluid within a subterranean formation is a function of fluid properties, and the various petrophysical properties of the rock (e.g., permeability, porosity, formation factor, elastic moduli, etc.), which may be determined as generally described above. In addition, chemical reactions at the fluid-solid interfaces within the formation over time alter the physical structure of the subterranean formation while also contributing to the overall fluid flow behavior within a subterranean rock formation. Attempts at modeling fluid flow within a subterranean rock formation have been made; however, there is a continuing need for improvements in such models, and particularly for models that account for the properties of the rock along with the above-mentioned chemical reactions in a 3D representation of the subterranean formation.

Accordingly, embodiments disclosed herein include systems and methods for modeling reactive transport within a subterranean formation. The term “reactive transport” as used herein refers to the transport of materials (e.g., dissolved components of the minerals) through porous media resulting in and from the occurrence of chemical reactions within the porous media. In some embodiments, the porous media comprises a subterranean formation, and the systems and methods described herein utilize digital images of rock samples along with modeled chemical reactions (e.g., via geochemical modeling software) in order to model reactive transport within a 3D representation of a rock sample of the subterranean formation. Thus, through use of the embodiments disclosed herein, more accurate predictions of structural changes (e.g., resulting from dissolution, precipitation, and reactive transport) and fluid flow may be made for porous media (e.g., a subterranean formation), which may further enhance various operations, such as hydrocarbon extraction, environmental mitigation, etc.

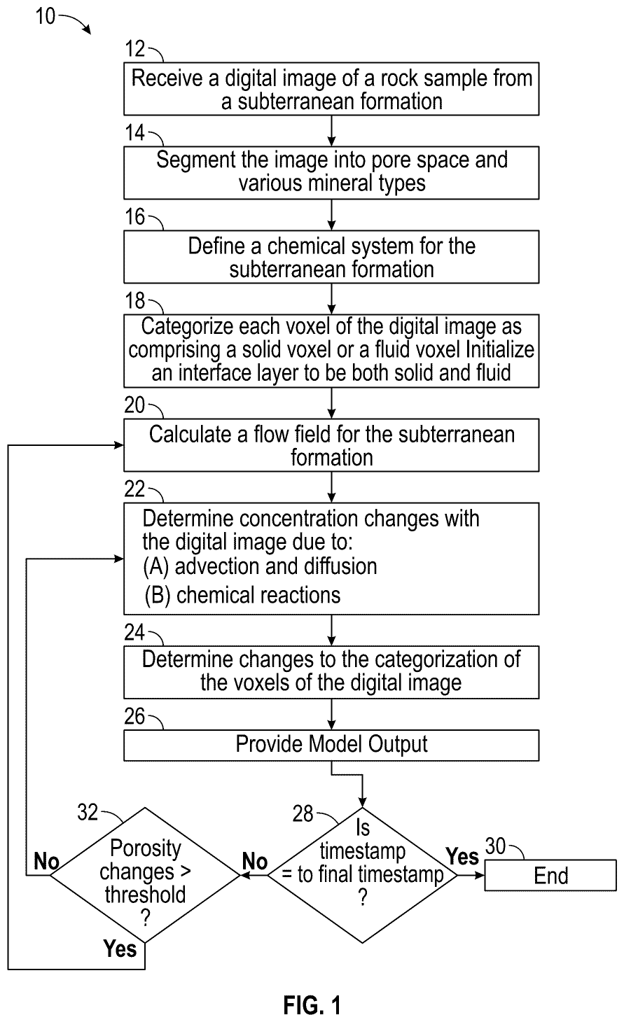

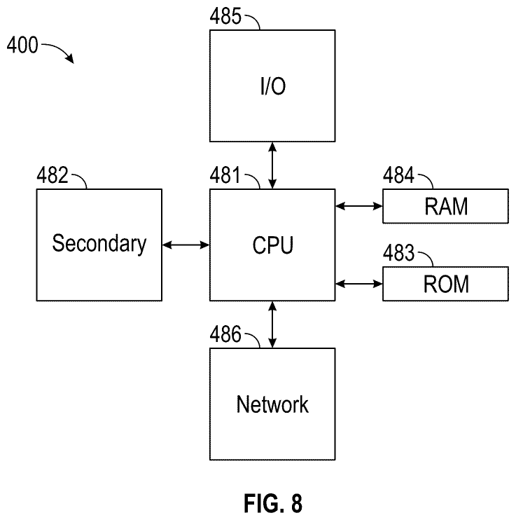

Referring now to FIG. 1, a method 10 for modeling reactive transport within a subterranean formation according to some embodiments is shown. In some embodiments, one or more features of method 10 may be performed by, with, or using a computer system or device (or multiple computer devices). The computer device (or devices) may comprise or be similar to the example computer system 400 shown in FIG. 8 and described below.

Initially, at block 12 method 10 comprises receiving a digital image (e.g., a 3D digital image) of rock from a subterranean formation. For instance, the digital image may comprise a digital image generated from a natural or synthetic rock sample, or other porous media. The rock sample (e.g., a natural rock sample) may comprise a rock sample from a subterranean formation of interest.

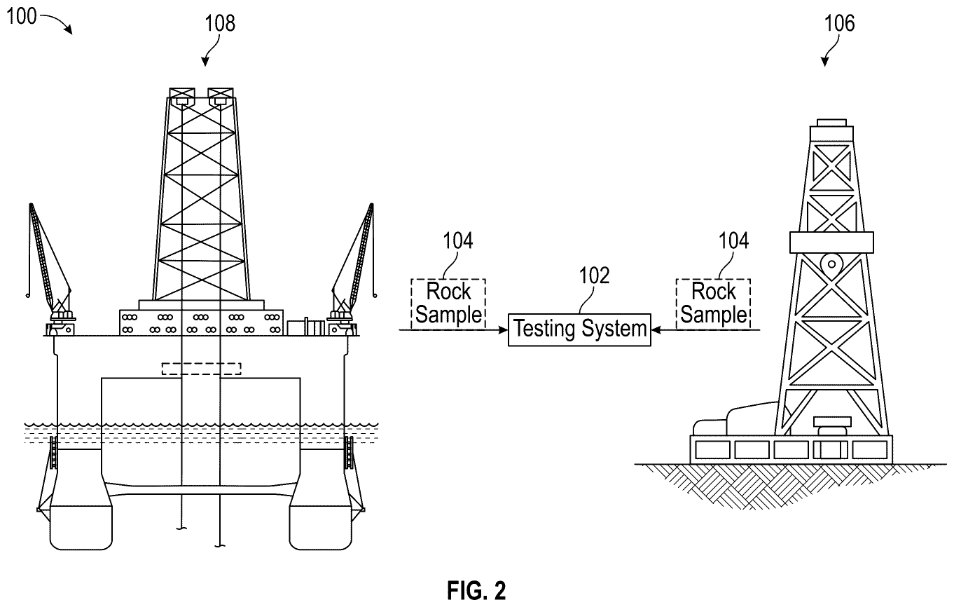

To provide an example, and referring to FIG. 2, a system 100 for acquiring and analyzing rock samples 104 for purposes of generating digital images of rock samples (e.g., the digital image received in block 12 of method 10 in FIG. 1) is shown according to some embodiments. In some embodiments, system 100 may be used to analyze rock samples 104 that are obtained from a subterranean formation for purposes of enhancing or facilitating oil and gas production from the subterranean formation. For example, in some embodiments rock samples 104 can be obtained from terrestrial drilling system 106 or from marine (ocean, sea, lake, etc.) drilling system 108, either of which is utilized to extract resources such as hydrocarbons (oil, natural gas, etc.), water, and the like.

The manner in which rock samples 104 are obtained and the physical form of those samples can vary widely. Examples of rock samples 104 useful in connection with embodiments disclosed herein include whole core samples, sidewall core samples (e.g., obtained from the sidewall of a wellbore extending through the subterranean formation), outcrop samples, drill cuttings, and laboratory generated synthetic rock samples such as sand packs and cemented packs.

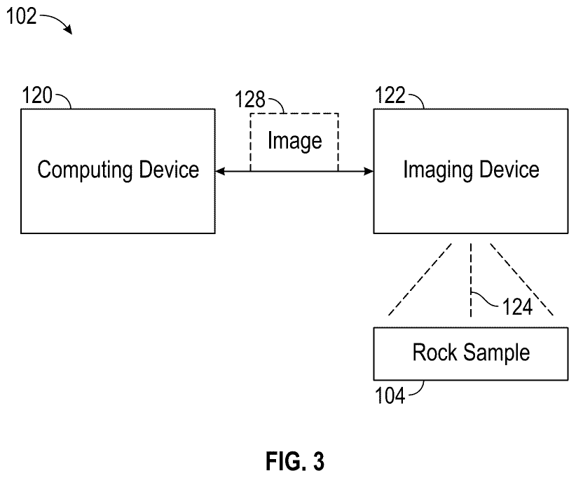

A testing system 102 of system 100 is configured to acquire and analyze 3D digital images 128 (shown in FIG. 3) of rock samples 104 in order to determine the physical properties of the corresponding sub-surface rock. The physical properties include, for example, petrophysical properties such as those petrophysical properties that are analyzed in the context of oil and gas exploration and production.

Referring to FIG. 3, a schematic diagram of testing system 102 is shown according to some embodiments. Testing system 102 includes imaging device 122 for obtaining 2D or 3D images, as well as other representations, of rock samples 104, such images and representations including details of the internal structure of the rock samples 104. An example of imaging device 122 is an X-ray CT scanner (or more simply “CT scanner”), which emits X-ray radiation 124 that interacts with an object and measures the attenuation of that X-ray radiation 124 by the object in order to generate an image of its interior structure and constituents. The particular type, construction, or other attributes of imaging device 122 can correspond to that of any type of X-ray device, such as a micro-CT scanner, capable of producing an image representative of the internal structure of rock sample 104. Other embodiments of imaging device 122 may utilize other imaging techniques, such as, X-ray nano-tomography, focused ion beam scanning electron microscopy, nuclear magnetic resonance, etc.

In some embodiments, the digital image volume may be computationally generated rather than produced by scanning the rock samples 104. In embodiments in which the digital image volume is produced by scanning a rock sample 104, the rock sample 104 may be a naturally occurring rock or a man-made porous material (e.g., a synthetic rock or other porous media).

3D digital image 128 may be composed of grayscale values representative of the attenuation of the X-ray radiation by the constituents of rock sample 104. In addition, 3D digital image 128 may comprise multiple two-dimensional (2D) slice images stacked along an axis of the rock sample 104, which together form the 3D digital image 128 of rock sample 104. In general, the stacking of the 2D slice images into 3D digital image 128 may be performed by computational resources of imaging device 122 itself, or by a separate computing device 120 from the series of 2D slice images 128 produced by imaging device 122, depending on the particular architecture of testing system 102. The computing device 120 may comprise or be similar to the computer system 400 shown in FIG. 8.

The images produced by imaging device 122 may be partitioned into 3D regular elements called volume elements, or more commonly “voxels”. For example, each voxel may be cubic, having a side of equal length in the orthogonal X, Y, and Z directions. In some embodiments, the images produced by the imaging device 122 may be partitioned into non-cubic grid volume elements having varying geometries. The 3D digital image 128 itself may contain different numbers of voxels in the X, Y, and Z directions. Each voxel within a respective 3D digital image has an associated numeric value, or amplitude, that represents the relative material properties of the imaged sample at that location of the medium represented by the digital volume. The range of these numeric values (commonly known as the grayscale range) depends on the type of digital volume, the granularity of the values, the size of the data register (e.g., 8-bit or 16-bit values), and the like. For example, 16-bit data values enable the voxels of an X-ray tomographic image volume to have amplitudes ranging from 0 to 65,536 with a granularity of 1. In some embodiments, testing system 102 may perform image enhancement techniques (e.g., data smoothing, noise reduction) on the 3D digital image. Likewise, it should be understood that other components of testing system 102 (e.g., imaging device 122 itself) may alternatively perform image enhancement in whole or in part. The 3D digital images 128 captured by the imaging device 122 may comprise the digital images received at block 12 of method 10.

Referring again to FIG. 1, method 10 includes, at block 14, estimating pore space and solid phase space in the rock sample using the digital image (e.g., the digital image obtained at block 12). In some embodiments, the testing system 102 shown in FIG. 2 performs segmentation or other image enhancement techniques on digital image 128 of rock sample 104 to distinguish and label different components or phases of digital image 128 shown in FIG. 3 from the grayscale values of digital image 128. More specifically, computing device 120 may perform segmentation to identify components, such as pore space and solid phase space (e.g., space comprising mineralogical components) in the digital image 128. The resulting 3D digital image has various solid material components labeled (for example, by mineral type) and the pore-space or void space labeled. In this exemplary embodiment, segmentation associates the voxels in the digital image with a particular material (or pore space, as the case may be) at the corresponding physical location within rock sample 104. Some or all of the voxels may be labeled with one or more material properties corresponding to the particular material constituent assigned to that voxel. For instance, such constituents may include pore space, matrix material, clay fraction, individual grains, grain contacts, mineral types, and the like.

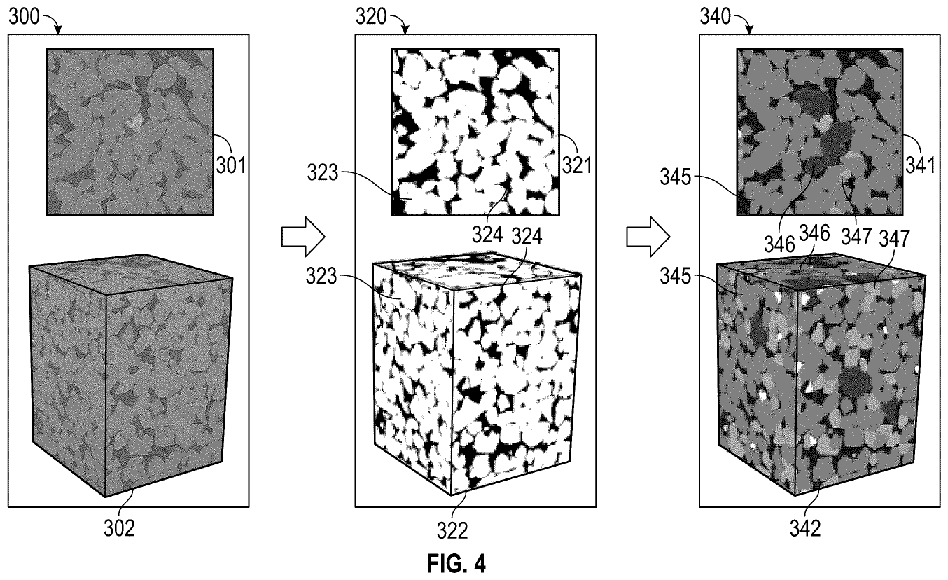

Referring to FIG. 4, exemplary views of digital image 128 of the rock sample 104 are shown. Particularly, FIG. 4 illustrates 2D slices 301, 320, 340 and associated 3D views 302, 322, 342 of the digital image 128. The 2D slice 301, and its associated 3D view 302 of the digital image 128 illustrates a cross-sectional slice of the structural details of rock sample 104, where the grayscale value is indicative of the X-ray attenuation of that voxel as attained by testing system 102. In some embodiments, computing device 120 (or another computing device or devices) may perform this segmentation to identify components, such as pore space and mineralogical components (e.g., clays and quartz). In some embodiments as represented in FIG. 4, the digital image 128 is segmented resulting in 2D slice 320, and associated 3D view 322 of the digital image, where each voxel is labeled as one, two or more significant phases, representing such material constituents as solid material 323 or pore space 324.

The computing device 120 may utilize any of a number of types of segmentation techniques to segment image 128. One approach to segmentation is the application of a “thresholding” process to image 128, in which computing device 120 chooses a threshold value within the voxel amplitude range. Those voxels having an amplitude below the threshold value are assigned one specific numeric value that denotes pore space, while those voxels having an amplitude above the threshold are assigned another numeric value that denotes matrix space (i.e., solid material). In this approach, thresholding converts a grayscale image volume to a segmented volume of voxels having different numeric values for example, 0, 1, 2, etc., depending on the number of labeled components.

Computing device 120 may utilize other segmentation techniques. For instance, in some embodiments computing device 120 may utilize Otsu's Method in which a histogram-based thresholding technique selects a threshold to minimize the combined variance of the lobes of a bimodal distribution of grayscale values (i.e., the “intra-class variance”). Otsu's method can be readily automated and extended to repeatedly threshold the image multiple times to distinguish additional material components such as quartz, clay, and feldspar. Other examples of automated segmentation techniques of varying complexity may alternatively or additionally be used by computing device 120 to distinguish different features of an image volume, such techniques including Indicator Kriging, Converging Active Contours, Watershedding, and the like.

In some embodiments and referring particularly to 2D slice 341 and associated 3D view 342 of image 128 shown in FIG. 4, the computing device 120 may further partition the identified phases of the segmented digital image 320 to further distinguish the solid components of image 128. Particularly, further segmentation may be applied one or more times to differentiate various features within the image to distinguish among different materials such as clay, quartz, feldspar, etc. based on different X-ray attenuation characteristics, chemical signatures from a scanning electron microscope, or other distinguishing features.

As an example, partitioning can be performed using any imaging software (e.g., Avizo™ Software available from ThermoFisher Scientific™ of Hillsboro, Oreg., USA). During partitioning, a group of voxels can be identified as a grain. That is, each voxel or group of voxels define or represent a single grain, and various grains may be represented by different colored (or pattern, shades of grey) voxel or group of voxels as associated to a phase as shown particularly in 2D slice 341 and 3D view 342 (e.g., phases 345, 346, and 347). Specifically, 2D slice 341 and 3D view 342 of digital image 128 illustrate an example of a partitioned image of rock sample 104 including structural details of rock sample 104. The size (e.g., area, volume, radius, etc.) of each grain may be determined via imaging software.

The resultant mineral-mapped digital image 340 is a 3D digital image in which each voxel is labeled by a value that corresponds either to pore-space or to a specific phase associated with a specific solid phase mineral (e.g., calcite, K-feldspar, quartz, anorthite, pyrite) depending on the geology of rock sample 104. In some embodiments, each voxel may be assigned an integer that can be mapped to a particular solid phase mineral or to pore-space. Specific solid phase minerals may correspond to multiple values in 3D view 342. In some embodiments, each of the voxels of 3D view 342 may be associated with a maximum of a single specific mineral to allow for the solving of the mass balance equation whereby the external geochemical equilibrium may be related to a solid phase change.

Referring back to FIG. 1, at block 16, method 10 includes defining a chemical system for the subterranean formation. The chemical system may comprise a collection of solid phase minerals (for example, those with corresponding labels in the mineral-mapped digital image 340 of FIG. 4), an in situ fluid composition, and an invading fluid composition. The solid phase minerals may be identified to be modeled in any number of ways, such as with a fast-equilibrium treatment or a full kinetic treatment. In some embodiments, a solid phase mineral may be treated thermodynamically, with an associated heat of dissolution, or the solid phase mineral may be treated isothermally, with no heat of dissolution. In some embodiments, a solid phase mineral may be treated as unreactive, where no chemical reaction is modeled in order to simplify the chemical system and/or reduce computational cost. In some embodiments, the chemical system defined at block 16 may define in situ fluid, which may be assumed to saturate the “fluid-phase” voxels of the digital image. The chemical system defined at block 16 may also define an “invading” fluid that may mix with the in situ fluid as it percolates through the rock. The chemical system defined at block 16 may also contain a defined heat capacity of the fluids and/or a function to compute the heat capacity based on the properties of a mixture of the fluids.

In some embodiments, block 16 includes initially modelling at least some (if not all) possible chemical reactions between interacting solid phase minerals and fluids (e.g., between the solid phase minerals and the solutes of the fluids) using geochemical modeling software or solvers. In some embodiments, the chemical reactions may be modeled using the PHREEQC geochemical modeling software provided by the United States Geological Survey (USGS). The chemical reactions may comprise chemical reactions that may occur at the solid/fluid interfaces (e.g., such as the reactions that occur between the fluids present in the pore space and solid chemical and/or mineral species that are present within the subterranean formation).

The chemical reactions identified at block 16 may comprise chemical reactions that potentially occur between the aqueous phase species within the subterranean formation and chemical reactions that potentially occur between the aqueous phase and mineral phase species within the formation. The chemical reactions between the aqueous phase species may be referred to here as “homogenous chemical reactions” and the chemical reactions between the aqueous and mineral phase species may be referred to herein as “heterogenous chemical reactions.” The solutes (e.g., ions such as H⁺, Ca⁻², CO₃⁻²) of the aqueous phase species may be tracked independently or simultaneously as lumped elemental groups.

In some embodiments, the type of reactions occurring within the subterranean formation depend not only on temperature and pressure conditions but also on the solid phase mineral composition of the formation and the characteristics of the fluid occupying the pore space such as its aqueous composition, ion solubility, and concentration. Several chemical reactions may be occurring simultaneously inside the subterranean formation, therefore the system of reactions considered for a given formation may be complex. However, in some embodiments, consideration may be limited to reactions having a significant effect on the pore space changes (reactions that result in precipitation or dissolution of material) or that impact the system in the time frame, pressure and temperature conditions of interest. In an effort to provide additional specific examples, Table 1 (shown below) includes some common reactions occurring in different subterranean formations such as sandstones, carbonates, and mudrocks.

TABLE 1

| | | Temperature |
|---|---|---|
| Process | Reaction | (° C.) |
| Quartz precipitation | H₄SiO₄ → SiO₂ + 2H₂O | >80 |
| Calcite precipitation | Ca²⁺ + CO₂ + H₂O → CaCO₃ + 2H⁺ | 100-160 |
| Albitization | Na⁺ + 3H₄SiO₄ + Al³⁺ → NaAlSi₃O₈ + 4H⁺ + 4H₂O | 75-200+ |
| | KAlSi₃O₈ + Na⁺ → K⁺ NaAlSi₃O₈ | |
| Pyrite precipitation | Fe²⁺ + 2H₂S → FeS₂ + 4H⁺ | — |
| K-feldspar and Kaolinite | KAlSi₃O₈ + Al₂Si₂O₅(OH)₄ → muscovite/illite + 2SiO₂ + H₂O | 50-120 |
| conversion | | |
| Organic matter | CH₂O + H₂O → CO₂ + 4H⁺ | 40-100 |
| Smectite to illite | smectite + Al³⁺ + K⁺ → illite + Si⁺⁴ | 60-100 |
| conversion | | |
| K-feldspar dissolution | KAlSi₃O₈ + 4H⁺ + 4H₂O → K⁺ + Al³⁺ + 3H₄SiO₄ | 80-160 |
| Carbonate dissolution | CaCO₃ + H⁺ → Ca²⁺ + HCO₃⁻ | >60 |
| Dolomite dissolution | CaMg(CO₃)₂ + 2H⁺ → Ca²⁺ + Mg²⁺ + 2HCO₃⁻ | 25-150 |
| Dedolomitization | CaMg(CO₃)₂ + CaSO₄ → 2CaCO₃ + MgSO₄ | <50 |
| Dolomitization | 2CaCO₃ + Mg²⁺ → CaMg(CO₃)₂ + Ca⁺ | 70-110 |

At block 18, method 10 includes mapping each of the voxels of the digital image 128 as “solid-phase” (i.e., containing solid material), “fluid phase” (i.e., containing fluid), or both. In some embodiments, all solid-phase or “solid” voxels must also be labeled with a maximum of one corresponding solid-phase mineral and a solid-phase mineral concentration (or amount of material in mols) such that each solid voxel has a defined composition including a solid-phase mineral of a subterranean formation. Initially, this mineral concentration may be equal to concentration converted from the density or the molar volume of the corresponding mineral so that the solid phase voxels begin with a “full” concentration. All fluid-phase or “fluid” voxels are assigned to have a zero solid phase mineral concentration, no solid phase mineral label is assigned, and one or more solute concentrations are assigned such that each fluid voxel has a defined fluid voxel composition including one or more solute components associated with a fluid of a subterranean formation. In some embodiments, the fluid voxels may be expanded (e.g., by a binary dilation or another method) to overlap with the “solid-phase” voxels. These voxels (hereafter referred to as “interface” voxels) may be understood as the overlap between the “solid-phase”and the “fluid-phase”, where the interface voxel has both properties of a “solid-phase” voxel (a solid phase concentration and mineral label) as well as “fluid-phase” properties (solute concentrations assigned). In some embodiments, the solid phase concentration may be reduced for the “interface” voxels so that at least initially are only half (or some other fraction) “full”.

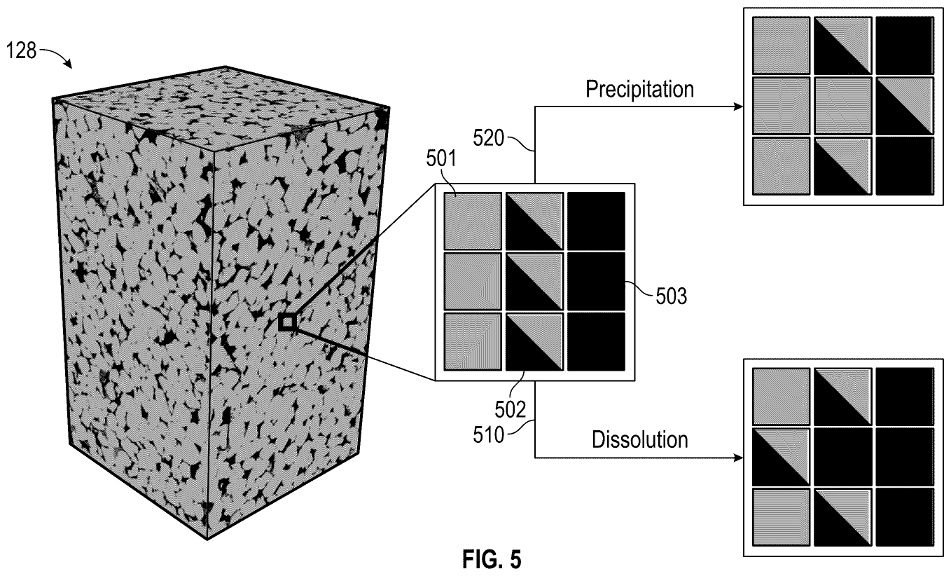

As an example, and referring briefly to FIG. 5, a portion of the digital image 128 is shown schematically as including a plurality of separate voxels. Particularly, FIG. 5 illustrates digital image 128 as including a plurality of solid voxels 501, a plurality of interface voxels 502, and a plurality of fluid voxels 503. As described above and as indicated in FIG. 5, the interface voxels 502 represent the overlap of the “solid-phase” and the “fluid-phase” where both solid and fluid properties may be tracked simultaneously. The spatial configuration of the plurality of interface voxels 502 defines an interface of the digital image 128 between the solid-phase and fluid-phase constituents thereof. The geometry of this interface may change over time (e.g., simulated time) through processes of dissolution 510 where one or more interface voxels 502 are converted into fluid voxels 503 and one or more solid voxels 501 are converted into interface voxels 502, and/or precipitation 520 where one or more interface voxels 502 are converted into solid voxels 501 and one or more fluid voxels 503 are converted into interface voxels 502.

Referring again to FIG. 1, method 10 includes, at block 20, identifying a flow field for the subterranean formation for all fluid voxels identified in block 18. In some embodiments, identifying the flow field for the subterranean formation comprises determining a distribution of pressure and fluid flow velocity (e.g., under steady state conditions) within the subterranean formation at a particular time stamp (e.g., an initial time stamp or a subsequent time stamp). In some embodiments, the distribution of pressure and fluid flow velocity may be computed using a LB based steady-state solution. The LB method is a temporally and spatially discrete solution to the Boltzmann equation that describes fluid flow and can fully recover the Navier-Stokes characterization of fluid behavior at the continuum scale. During this analysis, the fluid is modeled as a collection of discrete particles moving through a discretized regular lattice. In this lattice, the fluid is represented in terms of the probability of finding average populations of particles at a given time and position, with a certain velocity.

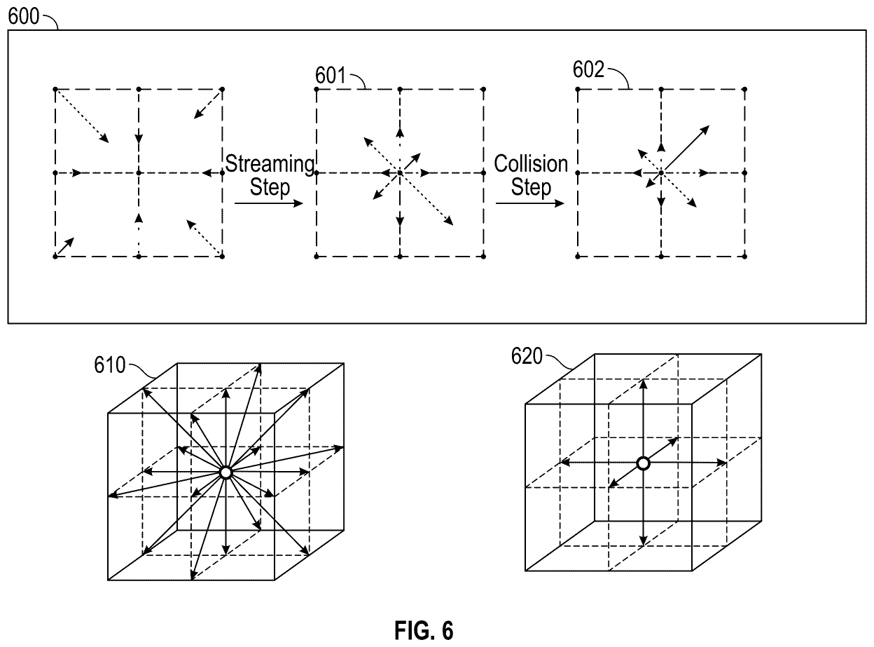

The particle movement through the lattice may be defined by a two-stage process occurring at each time step: streaming and collision. Referring to FIG. 6, an exemplary lattice 600 illustrating collision and streaming is shown. The streaming stage 601 refers to the advection of the particle to its next location (lattice site), and the collision stage 602 represents the interaction of the particle with other particles occupying this new location. The particles are limited to movement along a finite number of directions and the particle velocities are restricted to a specific number of discrete values. Both the model dimensions and the number of directions the particle is allowed to move determine the type of the given lattice.

Still Referring to FIG. 6, examples of 3D lattice site 610, 620 for use in determining the flow field are shown. The arrows indicate the various possible directions of movement for a particle. For example, lattice site 610 illustrates 19 velocity vectors including a zero velocity vector (e.g. non-movement position), and lattice site 620 illustrates 7 velocity vectors including a zero velocity vector. This approach provides information on macroscopic fluid properties and permits the simulation of complex geometries and surface configurations. Moreover, simple, and efficient parallel computation can be implemented using this methodology.

Returning again to FIG. 1, in some embodiments, method 10 is performed for a 3D representation (e.g., a 3D digital representation or image) of at least a portion of the subterranean formation. Thus, as alluded to above, the flow field identified in block 20 may comprise a 3D flow field that accounts for the distribution of pressure and fluid velocity along three orthogonal directions (e.g., x, y, and z) within the 3D representation of at least a portion of the subterranean formation.

Method 10 also includes determining concentration changes in the subterranean formation due to solute transport in block 22. Not intending to be bound by any particular theory, in some embodiments, the concentration changes in the 3D representation of the subterranean formation may be determined at block 22 according to Equation (5) presented below where Cₖ represents the concentration of the particular solute component (k), t represents time, u represents the flow rate, and D represents the diffusion coefficient, Rₘ represents the net change in concentration of species k due to chemical reaction with mineral m:

$$\frac{\partial {C}_{k}}{\partial t} = - ( u \cdot ∇ ) {C}_{k} + ∇ \cdot ( D \cdot ∇ {C}_{k} ) + {Σ}_{m} {R}_{m} \qquad ( 5 )$$

In addition, in Equation (5) ∇ can represent a number of different operators. For instance, for the term ∇Cₖ, ∇ represents the gradient of the concentration of species k (the rate of change in concentration in each direction) according to Equation (6) presented below:

$$∇ {C}_{k} = \frac{\partial {C}_{k}}{\partial x} + \frac{\partial {C}_{k}}{\partial y} + \frac{\partial {C}_{k}}{\partial z} \qquad ( 6 )$$

Conversely, for the term (u·∇) Cₖ in Equation (5), ∇ represents the rate of change in concentration but multiplied by the velocity in that direction according to Equation (7) presented below:

$$( u \cdot ∇ ) {C}_{k} = {u}_{x} \frac{\partial {C}_{k}}{\partial x} + {u}_{y} \frac{\partial {C}_{k}}{\partial y} + {u}_{z} \frac{\partial {C}_{k}}{\partial z} \qquad ( 7 )$$

Thus, within Equation (5) the expression “−(u·V) Cₖ” represents the solute movement in the 3D representation of the subterranean formation due the flow field (known as “advection”), the expression “∇·(D·∇Cₖ)” represents the solute movement in the 3D representation due to the flow field (known as “diffusion”), and the expression “ΣₘRₘ” represents the concentration changes in the 3D representation due to chemical reactions.

As used herein, the term “advection” refers to the movement of heat or matter due to fluid flow within a medium. Within a subterranean formation, fluid may be flowing due to pressure gradients present therein. As used herein, the term “diffusion” refers to the movement of heat or matter within a medium (e.g., a subterranean formation) from areas of high concentration to areas of lower concentration. Over time, both advection and diffusion may drive movement of the solute or heat within the subterranean formation (as modeled using the 3D representation) due to pressure and concentration gradients present within the formation.

In some embodiments, the concentration changes described in Equation (5) may be separated into two categories and solved independently: the physical movement due to advection and diffusion of fluid solutes (e.g., solute transport) modeled in part “(A)” of block 22, and the chemical consumption or production of solutes by chemical reactions (e.g., both homogeneous and heterogenous reactions) modeled at part “(B)” of block 22. This separation of concentration changes in the subterranean formation due to solute transport from concentration changes in the subterranean formation due to chemical reactions is known as an operator splitting, but the concentration changes due to both solute transport and chemical reactions may be alternatively determined in a single step combining (A) and (B) of block 22.

Specifically, in some embodiments, Equation (5) can be solved at block 22 using a multi-component reactive transport model at the pore-scale. In some embodiments, the model combines using a modified LB solution that recovers pore-scale fluid transport in terms of advection and diffusion, with a reactive component that accounts for homogeneous reactions assumed to be in a local thermodynamic equilibrium and heterogeneous reactions treated via a kinetic, pseudo-equilibrium, or some other model (e.g., via PHREEQC geological modeling software).

In some embodiments, the changes in concentration due to solute transport may be analyzed by considering solute transport that occurs due to both advection and diffusion of the fluids within the 3D representation of the subterranean formation. In some embodiments, the advection and diffusion processes may be modeled simultaneously with another LB solver. For example, and referring briefly to FIG. 6, these processes may be accurately modeled using 3D lattice 620. Unlike the flow field LB solution at block 20 of method 10, the modified LB solution 620 provides information about the concentration changes for a given k^{th} chemical component (not the velocity field of the composite fluid) in terms of a distribution function (G).

Not intending to be bound by any particular theory, the general form of the modified distribution function may be expressed in accordance with Equation (8) presented below where Gₐₖ represents the distribution function in an a direction in the velocity space for the k^{th} component, δt represents the time increment, eₐ represent the velocity vectors, Gₐₖ^{eq} represents the equilibrium distribution function, u represents the velocity, Cₖ represents the concentration of the k^{th} component, and τ_{aq} represents the dimensionless relaxation time:

$${G}_{α k} ( x + {e}_{α} δ t , t + δ t ) = {G}_{α k} ( x , t ) - \frac{{G}_{α k} ( x , t ) - {G}_{α k}^{eq} ( {C}_{k} , u )}{{τ}_{aq}} \qquad ( 8 )$$

The equilibrium distribution function, velocity vectors, and relaxation time are dependent on the lattice model (e.g., it is a function of the lattice dimensions and the number of lattice velocities). Not intending to be bound by any particular theory, for a three-dimensional seven-velocity lattice 620, the form of the equilibrium distribution function is shown in Equation (9), and the relaxation time in Equation (10) presented below:

$${G}_{α k}^{eq} ( {C}_{k} , u ) = ( 1 + \frac{{e}_{α} u}{{e}_{s}^{2}} + \frac{{e}_{α} {e}_{α} : uu}{2 {e}_{s}^{4}} - \frac{u \cdot u}{{e}_{s}^{2}} ) {ω}_{α} {C}_{k} ( x , t ) \qquad ( 9 )$$

$${τ}_{aq} = \frac{1}{2} \partial t + \frac{D}{{e}_{s}^{2}} \qquad ( 10 )$$

In Equation (9) and Equation (10), eₛ is the pseudo lattice speed of sound and ωₐ are weight coefficients. Both parameters depend on the lattice type. In addition, in Equation (9) and Equation (10), D is the diffusion coefficient.

The number of distribution functions utilized to capture the concentration changes in each voxel of the 3D representation of the subterranean formation is given by the number of lattice velocities and the chemical species involved in the system as previously described (block 20 in FIG. 1). Not intending to be bound by any particular theory, once the distribution functions are calculated for every lattice velocity and component, the concentration of a given k^{th} component (Cₖ) can be estimated through the summation of the distribution functions over all discrete velocities as shown in Equation (11) presented below.

$${C}_{k} = {Σ}_{α} {G}_{α k} \qquad ( 11 )$$

Returning to FIG. 1, in some embodiments, (A) of block 22 includes modeling the transport of the ion concentrations via advection and diffusion for all fluid voxels identified at block 16. In some embodiments, the lumped elemental concentrations of the solute species are tracked instead of the concentrations of each solute species, thereby reducing the computational cost and enhancing computational efficiency.

Following part (A) of block 22, method 10 also includes modeling concentration changes due to chemical reactions at part (B) of block 22. In some embodiments, the concentration changes due to chemical reactions may describe the interactions of components within the fluid or aqueous phase (i.e., homogenous reactions) and between the fluid and the solid phase minerals (i.e., heterogenous reactions) within the 3D representation of the subterranean formation. At least some of the chemical reactions may comprise at least some of the collection of chemical reactions identified at block 16. Additionally, in some embodiments, the reactive component of Equation (5) accounts for homogeneous and heterogeneous reactions.

Both homogeneous and heterogenous reactions may, for example, be characterized based on the assumption of local thermodynamic equilibrium and using the mass action law as shown in Equation (12) presented below. In certain embodiments, geochemical modeling software (e.g., PHREEQC) may be employed to determine concentration changes arising from homogenous and pseudo-equilibrium reactions.

$${C}_{i} = {( {γ}_{i} )}^{- 1} {K}_{i} {\prod }_{j = 1}^{{N}_{C}} {( {γ}_{j} {C}_{j} )}^{{v}_{ji}} \qquad ( 12 )$$

Within Equation (12) the primary species (Cⱼ) and secondary species concentrations (Cᵢ) are related as a function of the number of independent species associated to primary species (N_{c}) using the stoichiometric coefficients of homogeneous reactions (νⱼᵢ), the activity coefficients (γⱼ and γᵢ) and the equilibrium constant (Kᵢ).

On the other hand, heterogeneous reactions at the solid-fluid interface may instead be treated kinetically by considering the reaction rates of the chemical species of interest as shown in Equation (13) presented below. A general form of kinetic rate law applied at the interface to account for heterogeneous reactions is shown in Equation (13), but in general any rate law may be used to model the kinetic rate of a heterogenous reaction.

$$D \frac{\partial {Ψ}_{k}}{\partial n} = {\sum }_{m = 1}^{{N}_{m}} {v}_{km} {I}_{m}^{*} \qquad ( 13 )$$

Within Equation (13) presented above, n represents the direction normal to the solid/fluid surface, νₖₘ represents the stoichiometric coefficient of the reaction, Nₘ represents the number of reactive minerals (number of heterogeneous reactions), and I*ₘ represents the reaction rate of the solid phase mineral which is dependent on the concentration. As an example, geochemical modeling software may allow a kinetic treatment to be toggled on or off for specific mineral types such that a user may choose a kinetic treatment for a given mineral type or to instead use the local equilibrium assumption.

Note that in some embodiments where the lumped component concentrations are tracked rather than the concentration of each solute species, the homogeneous reactions are inconsequential to the lumped component concentrations and may be excluded to minimize the computing power required. Indeed, if the lumped component concentrations are tracked, only the heterogeneous reactions need to be modeled, and therefore the chemical reactions only need to be modeled at the interface voxels that contain both “solid-phase” and “fluid-phase”.

For heterogenous reactions, the change in solid phase mineral concentration of the interface voxel may be determined based on a geochemical calculation (performed independently for each interface voxel of the 3D representation) performed between the solid phase mineral and the fluid associated with the respective interface voxel. In some embodiments, the geochemical calculations may be implemented using a computer-implemented geochemical modeling tool such as, for example, the PHREEQC geochemical modeling software package. Changes in the solute concentrations of the fluid of the interface voxel may be converted into changes in the solid phase mineral concentration specifically associated with the interface voxel by using known stochiometric relationships between the solid phase mineral and the one or more of the solutes in the fluid. In some embodiments, changes in the concentration of solutes in the fluid and solid phase minerals within the subterranean formation may be modeled or predicted based on the concentration changes in the 3D representation determined at block 22. Not intending to be bound by any particular theory, in some embodiments, the heat released or consumed by the change in the concentrations of the solid phase mineral may be modeled and converted to a change in temperature as shown in Equation (14).

$$Δ {H}_{rxn} = {nC}_{P} Δ T \qquad ( 14 )$$

Within Equation (14), n is the number of mols of reaction (e.g., the change of concentration in solid phase mineral), C_{P} is the heat capacity of the fluid which may be provided by the chemical system definition in block 16, ΔH_{r×n} is the enthalpy of the dissolution reaction which may be provided by the chemical system definition in block 16, and ΔT is the change in temperature associated with the reaction. For certain mineral types, the reactions may be treated isothermally by setting ΔH_{r×n} to zero.

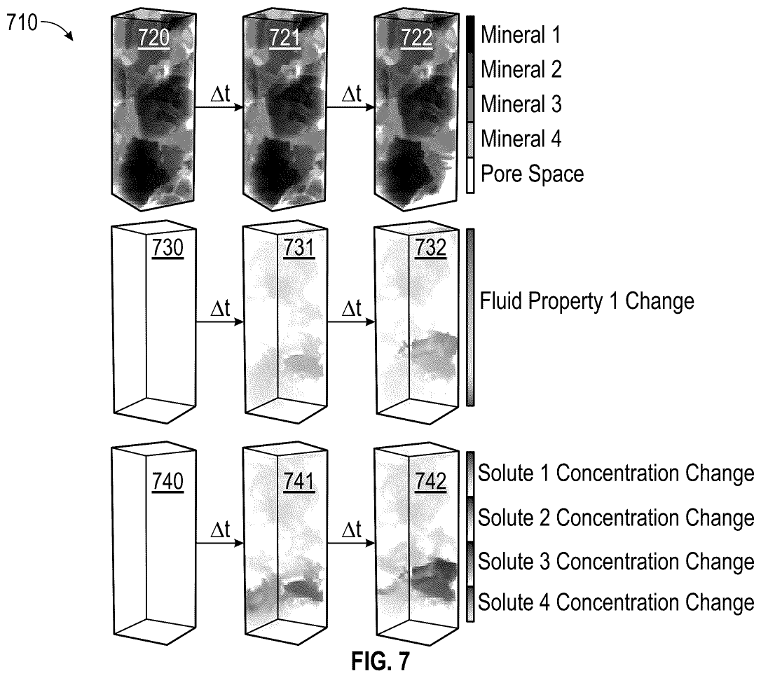

Referring now to FIG. 7, an exemplary depiction of an application of a solution 710 to Equation (5) as described above is shown schematically for a select few time stamps. The solution 710 may use a mineral labeled 3D digital representation of rock sample 104 as an input. For the solid voxels, the solid mineral concentration is tracked as well as the mineral label 720. For the fluid voxels, fluid properties 730 (e.g, pH, temperature, pe) and the concentrations of all chemically active solutes 740 are tracked as they evolve under Equation (5). While only a few chemically solutes are shown, it should be understood that many more chemically active solutes may be present in the simulation that can be adequately shown in FIG. 7. Similarly, while only one fluid property is shown, in should be understood that many more fluid properties may be tracked in the simulation. As the fluid chemically reacts with the digital representation of rock sample 104 at each timestamp, the pore-space geometry is updated (721-722) to reflect dissolution and precipitation of the solid phase under the chemical conditions of the fluid at each voxel, the fluid properties are updated (731-732), and the solute concentrations are updated (741-742), as described above.

Referring again to FIG. 1, at block 24 method 10 includes determining changes to the categorization of the voxels of the 3D representation or digital image (e.g., digital image 128 shown in FIG. 3), since the solid mineral concentration was updated in block 22. In some embodiments, block 22 only includes changes to the mineral concentrations in the “interface” voxels that contain both solid phase and fluid phase properties.

For instance, and briefly referring to FIGS. 1 and 5, an interface voxel 502 may initially have a non-zero solid phase mineral concentration, but after the update in block 20, it may now contain zero solid phase mineral concentration. In this instance, the solid phase mineral could be said to have fully dissolved, the voxel is removed from the “solid-phase” mask, and the mineral label is removed. Since the voxel no longer contains solid phase mineral properties and contains only fluid phase properties, it is also no longer considered to be an interface voxel. In order to keep an active “interface” layer of voxels (so that reactions continue to occur), all nearest neighbors of the dissolved voxels are added to the “fluid-phase” mask and thereby become interface voxels as shown in arrow 510. In some embodiments, the solute concentrations of each new fluid voxel are initialized to be the average respective solute concentration of its nearest fluid neighbors.

Conversely, an interface voxel 502 may initially have a solid phase mineral concentration less than the density of its assigned mineral type, but after the update at block 20, it may contain a solid phase mineral concentration greater than or equal to the mineral density. In this instance, the voxel could be said to be fully precipitated since there is no more room for fluid phase. In this instance, the voxel is removed from the “fluid-phase” mask, the solute concentrations are set to zero, and solid phase concentration is set to the density of the “full” voxel. Since this voxel no longer contains fluid phase properties and contains only solid phase mineral properties, it is no longer considered to be an interface voxel. In order to keep an active “interface” layer of voxels (so that reactions continue to occur), all nearest neighbors of the precipitated voxels are added to the to the “solid phase” mask and thereby become interface voxels as shown in arrow 520. In some embodiments, the new interface voxels are assigned the mineral phase of the fully precipitated voxel and the solid phase mineral concentration is initialized to zero since there is not yet any solid phase present.

After the pore-space geometry and the fluid concentrations in the digital image are updated, method 10 proceeds to block 28 where the model data output is captured. In some embodiments, the model output at block 26 may comprise the configuration of the digital image at the given time stamp of method 10 in terms of a configuration of the pore-space geometry, solute concentrations at all voxels, the porosity, the permeability, and any other property of the digital image. The model output at block 26 may be output at each iteration of the cycle in method 10 or only when the time stamps matches a particular criteria (e.g., every 1000 steps through the cycle). Thus, the model output may show a prediction of a progression or change in pore-space geometry, material concentrations (e.g., concentrations of solid phase minerals and/or solute concentrations), or physical properties (e.g., porosity and/or permeability) within the 3D representation of the subterranean formation over a period of time (e.g., at a plurality of discrete time stamps). Such a prediction may be useful for predicting reactive transport within the subterranean formation during mineral (e.g., hydrocarbon) extraction operations or for other operations (e.g., contaminant tracking operations). The model output provided at block 26 may be provided or displayed to a user and/or may be saved to a memory device for subsequent analysis.

At block 28, following the providing of a model output at block 26, method 10 includes determining whether the current time stamp is equal to the final time stamp. At the initiation of method 10, a finite number of time stamps may be determined. The time stamps may correspond to the period of time (e.g., a period of days, months, years, or number of time stamps computed within method 10, etc.) that are being considered for simulating the subterranean formation. Thus, the number of time stamps may vary widely for each performance of the method. However, if blocks 22, 24, and/or 26 have been performed for the predetermined number of time stamps at block 28, the determination may be “yes,” so that method 10 may progress to block 30 ending the simulation at block 30. In some embodiments, block 30 may include, along with ending the simulation, providing a final model output corresponding to the final time stamp. If, on the other and, the time stamp at block 28 is not the final time stamp, the determination at block 28 may be “no” so that the time stamp is incremented and the method 10 continue to block 32.

Because the fluid voxels are being dynamically updated at block 24 due to the chemical reactions determined in block 22, the fluid flow field calculated in block 20 needs to be updated to reflect the new pore-space geometry. In some embodiments, this update to the fluid flow field is only performed periodically to reduce the required computational power, because pore-space geometry may change slowly, or because the fluid field may react slowly to the changing pore-space geometry. At block 32, method 10 includes a determination of whether the fluid field needs to be updated. In some embodiments, this depends on whether a porosity change across the digital image has exceeded a predefined threshold for the given time step of method 10. In some embodiments, the porosity may be defined to be the fraction of solid voxels in the 3D digital representation of the digital image. The predefined threshold may be set by a user in some embodiments prior to the execution of method 10. This threshold may represent a comparison (e.g., a percent difference) between the current porosity and the porosity of the digital image when the last fluid flow field was calculated in block 20.

In this exemplary embodiment, if the porosity change for the given time stamp exceeds the predefined threshold, the determination at block 32 may be “yes” so that the method 10 may recycle back to block 20 to initiate performance of blocks 20, 22, 24, and 26 for an additional timestep. In this manner, the flow field (block 20) may be redefined or recalculated to reflect the new pore-space geometry of the digital image. If, on the other hand, the porosity change for the given time stamp does not exceed the predefined threshold, the determination at block 32 may be “no” so that the method 10 may recycle back to block 22 to initiate performance of blocks 22, 24, and 26 for an additional timestamp.

The embodiments disclosed herein include systems and methods for modeling reactive transport within a subterranean formation as a function of time. In some embodiments, the systems and methods described herein utilize digital images of rock samples along with a defined set of chemical reactions at the fluid-solid interfaces in order to generate a model of the fluid flow behavior within a 3D representation of the subterranean formation as a function of time. Thus, through use of the embodiments disclosed herein, more accurate predictions of fluid flow may be made for a subterranean formation, which may further enhance various operations, such as hydrocarbon extraction, environmental mitigation, etc.

Any of the systems and methods disclosed herein can be carried out (e.g., entirely or partially) on a computer or other device comprising a processor (e.g., a desktop computer, a laptop computer, a tablet, a server, a smartphone, or some combination thereof). Referring now to FIG. 8, a computer system 400 suitable for implementing one or more embodiments disclosed herein is shown. The computer system 400 includes a processor 481 (which may be referred to as a central processor unit or CPU) that is in communication with memory devices including secondary storage 482, read only memory (ROM) 483, random access memory (RAM) 484, input/output (I/O) devices 485, and network connectivity devices 486. The processor 481 may be implemented as one or more CPU chips.

It is understood that by programming and/or loading executable instructions onto the computer system 400, at least one of the CPUs 481, the RAM 484, and the ROM 483 are changed, transforming the computer system 400 in part into a particular machine or apparatus having the novel functionality taught by the present disclosure. Thus, the RAM 484 and/or the ROM 483 may comprise a non-transitory machine-readable (or computer-readable) medium that may include instructions (which may be referred to herein as machine-readable instructions) that are executable by CPU 481 to provide functionality to computer system 400. Thus, in some embodiments, a machine-readable instructions stored on a memory may be executed on a processor, so as to configured the processor to carry out some or all of the features of the methods described herein (e.g., method 10).

It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware (for example in an application specific integrated circuit (ASIC), or field-programmable gate arrays (FPGA)) because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the system 400 is turned on or booted, the CPU 481 may execute a computer program or application. For example, the CPU 481 may execute software or firmware stored in the ROM 483 or stored in the RAM 484. In some cases, on boot and/or when the application is initiated, the CPU 481 may copy the application or portions of the application from the secondary storage 482 to the RAM 484 or to memory space within the CPU 481 itself, and the CPU 481 may then execute instructions of which the application is comprised. In some cases, the CPU 481 may copy the application or portions of the application from memory accessed via the network connectivity devices 486 or via the I/O devices 485 to the RAM 484 or to memory space within the CPU 481, and the CPU 481 may then execute instructions of which the application is comprised. During execution, an application may load instructions into the CPU 481, for example load some of the instructions of the application into a cache of the CPU 481. In some contexts, an application that is executed may be said to configure the CPU 481 to do something, e.g., to configure the CPU 481 to perform the function or functions promoted by the subject application. When the CPU 481 is configured in this way by the application, the CPU 481 becomes a specific purpose computer or a specific purpose machine.

The secondary storage 482 is typically comprised of one or more disk drives or tape drives and is used for non-volatile storage of data and as an over-flow data storage device if RAM 484 is not large enough to hold all working data. Secondary storage 482 may be used to store programs which are loaded into RAM 484 when such programs are selected for execution. The ROM 483 is used to store instructions and perhaps data which are read during program execution. ROM 483 is a non-volatile memory device which typically has a small memory capacity relative to the larger memory capacity of secondary storage 482. The RAM 484 is used to store volatile data and perhaps to store instructions. Access to both ROM 483 and RAM 484 is typically faster than to secondary storage 482. The secondary storage 482, the RAM 484, and/or the ROM 483 may be referred to in some contexts as computer readable storage media and/or non-transitory computer readable media.

I/O devices 485 may include printers, video monitors, electronic displays (e.g., liquid crystal displays (LCDs), plasma displays, organic light emitting diode displays (OLED), touch sensitive displays, etc.), keyboards, keypads, switches, dials, mice, track balls, voice recognizers, card readers, paper tape readers, or other well-known input devices.

The network connectivity devices 486 may take the form of modems, modem banks, Ethernet cards, Omni-Path Architecture (OPA), InfiniBand (IB), universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identity (RFID), and/or other air interface protocol radio transceiver cards, and other well-known network devices. These network connectivity devices 486 may enable the processor 481 to communicate with the Internet or one or more intranets. With such a network connection, it is contemplated that the processor 481 might receive information from the network, or might output information to the network (e.g., to an event database) in the course of performing the methods described herein. Such information, which is often represented as a sequence of instructions to be executed using processor 481, may be received from and outputted to the network, for example, in the form of a computer data signal embodied in a carrier wave.

Such information, which may include data or instructions to be executed using processor 481 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several known methods. The baseband signal and/or signal embedded in the carrier wave may be referred to in some contexts as a transitory signal.

The processor 481 executes instructions, codes, computer programs, scripts which it accesses from hard disk, floppy disk, optical disk, solid state drives (SSD) (these various disk-based systems may all be considered secondary storage 482), flash drive, ROM 483, RAM 484, or the network connectivity devices 486. While only one processor 481 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 482, for example, hard drives, floppy disks, optical disks, and/or other device, the ROM 483, and/or the RAM 484 may be referred to in some contexts as non-transitory instructions and/or non-transitory information.

In an embodiment, the computer system 400 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 400 to provide the functionality of a number of servers that is not directly bound to the number of computers in the computer system 400. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid-state memory chip, for example analog magnetic tape, compact disk read only memory (CD-ROM) disks, floppy disks, jump drives, digital cards, multimedia cards, and others. The computer program product may be suitable for loading, by the computer system 400, at least portions of the contents of the computer program product to the secondary storage 482, to the ROM 483, to the RAM 484, and/or to other non-volatile memory and volatile memory of the computer system 400. The processor 481 may process the executable instructions and/or data structures in part by directly accessing the computer program product, for example by reading from a CD-ROM disk inserted into a disk drive peripheral of the computer system 400. Alternatively, the processor 481 may process the executable instructions and/or data structures by remotely accessing the computer program product, for example by downloading the executable instructions and/or data structures from a remote server through the network connectivity devices 486. The computer program product may comprise instructions that promote the loading and/or copying of data, data structures, files, and/or executable instructions to the secondary storage 482, to the ROM 483, to the RAM 484, and/or to other non-volatile memory and volatile memory of the computer system 400.

In some contexts, the secondary storage 482, the ROM 483, and the RAM 484 may be referred to as a non-transitory computer readable medium or a computer readable storage media. A dynamic RAM embodiment of the RAM 484, likewise, may be referred to as a non-transitory computer readable medium in that while the dynamic RAM receives electrical power and is operated in accordance with its design, for example during a period of time during which the computer system 400 is turned on and operational, the dynamic RAM stores information that is written to it. Similarly, the processor 481 may comprise an internal RAM, an internal ROM, a cache memory, and/or other internal non-transitory storage blocks, sections, or components that may be referred to in some contexts as non-transitory computer readable media or computer readable storage media.

Simulating reactive transport within 3D porous media can become computationally expensive given the size of the relevant domain and the complexity of the chemical system of the porous media. Indeed, the computational expense associated with such simulations may limit their capabilities and applicability with respect to different applications. Thus, techniques for maximizing the computational efficiency of such simulations may valuably enhance the utility of such simulations while broadening their applicability to applications which previously may not have been practical with less efficient techniques.

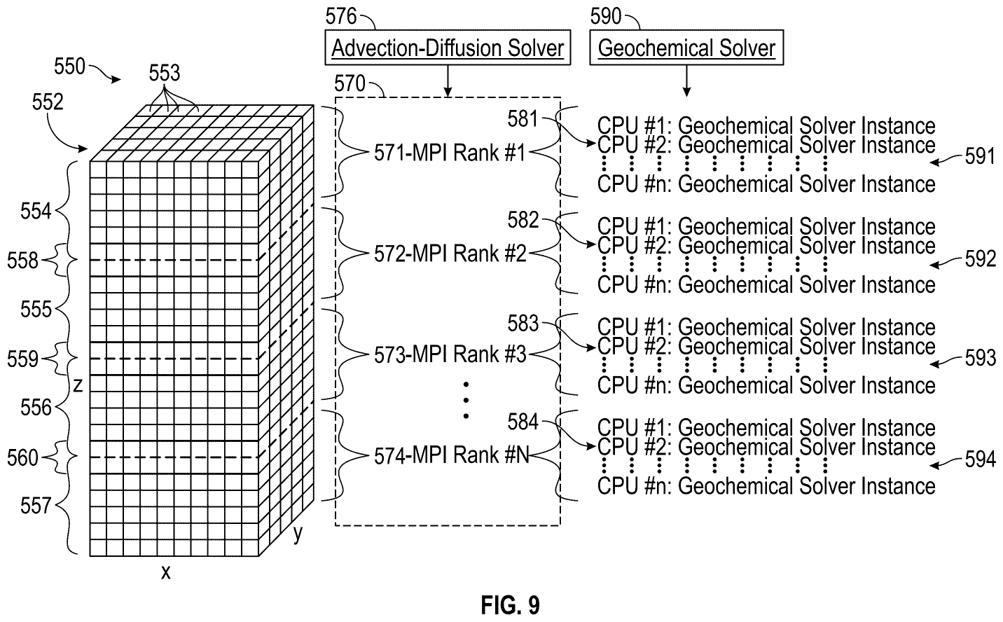

Referring now to FIG. 9, a schematic depiction of a parallelism scheme (method 550) for simulating geochemical changes in a three-dimensional digital image 552 representing a porous media is shown. In this exemplary embodiment, digital image 552 is divided into a plurality of distinct domains, hereafter called “chunks” (e.g., N chunks). In this example, digital image 552 is divided into chunks 554, 555, 556, and 557 distributed contiguously along a Z-dimension, where each chunk 554-557 comprises a plurality of separate voxels 553. Additionally, in this exemplary embodiment, overlapping (along the Z-dimension) halo regions 558, 559, and 560 are defined at the boundaries between the chunks 554, 556, and 557. Particularly, in this exemplary embodiment, a first halo region 558 extends along the Z-dimension overlapping a first chunk 554 and a second chunk 555, a second halo region 559 extends along the Z-dimension overlapping second chunk 555 and a third chunk 556, and a third halo region 560 extends along the Z-dimension overlapping the third chunk 556 and a fourth chunk 557.

In some embodiments, one or more of the chunks 554-557 may contain solid voxels, fluid voxels, and/or interface voxels where both fluid and solid exist. While the digital image 552 of FIG. 9 is shown as comprising four roughly equally sized chunks 553-557, in other embodiments, the number of chunks defining a given digital image (e.g., digital image 552) may vary. For instance, in other embodiments, digital image 552 may be divided into two chunks, three chunks, or more than four chunks. In other embodiments, the sizes of the chunks of a given digital image (e.g., chunks 554-557 of digital image 552) may vary in size as needed to balance the workload across a given computing system. In other embodiments, the chunks of a given digital image (e.g., chunks 553-557 of digital image 552) may be arranged in configurations which vary from that shown in FIG. 9. For instance, in another embodiment, chunks 554-557 may be arranged along the Y-direction of FIG. 9, the X-direction of FIG. 9, or combinations of the X, Y, and Z-directions of FIG. 9.

Additionally, in this exemplary embodiment, a message passing interface (MPI) program 570 comprising a plurality of separate initialized MPI processes is used to decompose the domain 552 into chunks 554-557. Particularly, each chunk 553-557 is assigned to a separate MPI process or instance identified by their respective separate ranks 571-574. For example, given N MPI instances of a program, a digital image may be divided into N chunks of data, each chunk assigned to a different MPI process. That is, a specific MPI process is assigned a separate chunk, such that multiple MPI processes may execute simultaneously and operate on different chunks of data. Using the assigned MPI ranks, the MPI program may differentiate which chunks of the applied data should be read by which process of the MPI program, and how to communicate results across the boundaries of the given MPI ranks (e.g., MPI rank 574 shown in FIG. 9 may provide MPI rank 573 changes made to its top layer while MPI rank 573 provides MPI rank 574 the changes made to its bottom layer so that there is a continuous flow across the boundaries).

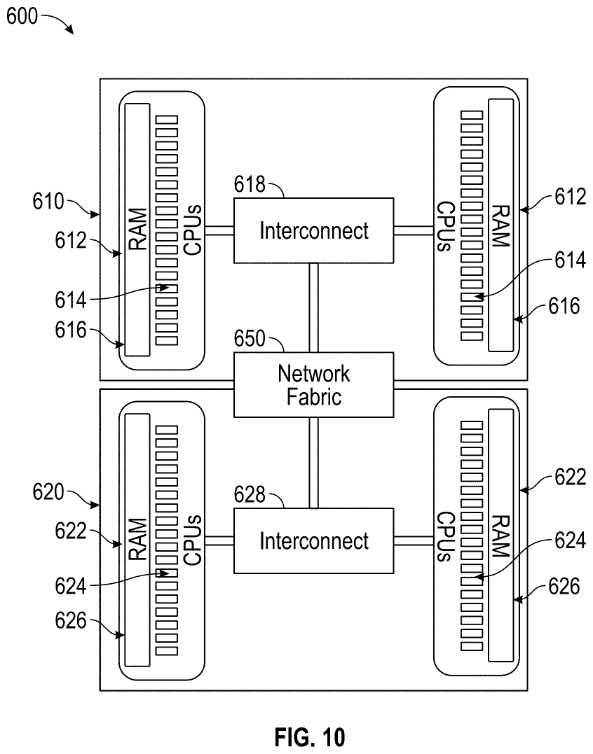

Referring briefly to FIG. 10, an example of a computer architecture 600 for use in a parallelism scheme is shown. In this example, two separate computer devices or nodes 610, 620 are shown. In this exemplary embodiment, each computer node; 610, 620 comprises a pair of sockets 612 and 622, respectively. In some embodiments, each socket 612 and 622 comprises one or more central processing units (CPUs) 614 and 624. As used herein, a CPU core refers to one or more transistors positioned on a single silicon wafer capable of performing floating point operations on one or more registers based on an input instruction provided to the respective CPU core. The CPU cores 614 and 624 of sockets 612 and 622 may perform separate floating-point operations in parallel.

In addition to CPU cores 614 and 624, in this exemplary embodiment, each socket 612 and 622 also comprises a memory device 616 and 626, respectively. Particularly, memory devices 616 and 626 each comprise random access memory (RAM) devices such as, for example, Double Data Rate (DDR) RAM. In this exemplary embodiment, each CPU 614 and 626 may access the entirety of the corresponding memory device 616 and 626 of the socket 612 and 624, respectively.

Although FIG. 10 illustrates a pair of computer nodes 610 and 620 each having a pair of sockets 612 and 622, it should be noted that the number of computer nodes (e.g., number of computer nodes 610 and 620), sockets (e.g., number of sockets 612 and 622), CPU cores (e.g., number of CPU cores 614 and 624), and memory present in a given computer architecture may vary depending on the requirements of the given application, and thus the configuration of computer architecture 600 should be construed generally.

In this exemplary embodiment, computer architecture 600 additionally comprises an internode interconnect, e.g., network fabric 650 providing a communication protocol to facilitate communication between the separate computer nodes 610 and 620 to enable parallel computing between the pair of computer nodes 610 and 620, and an intranode interconnect, e.g., interconnects 618, 628; providing a communication protocol to facilitate communication between the sockets 612 of computer node 610 and between the sockets 622 of computer node 620, respectively. In other words, intranode interconnect 618 facilitates communication (e.g., enabling parallel computing) between the sockets 612 of computer node 610 while intranode interconnect 628 facilitates communication (e.g., enabling parallel computing) between the sockets 622 of computer node 620. The network fabric 650 may comprise an Ethernet architecture or other communication architectures such as, for example, InfiniBand and Omni-Path communication architectures.

The intranode interconnects 618, 628 of computer nodes 610 and 620, may be hardwired onto a motherboard of the given computer node (e.g., motherboards of computer nodes 610, 620) depending on the given computer architecture. In some embodiments, each of the plurality of CPU cores of a given socket of a computer node may access to the entirety of the memory device of other sockets of the computer node via the intranode interconnect of the computer node. As an example, the CPU cores 614 of a first socket 612 of the computer node 610 may access the entirety of the memory device 616 of a second socket 612 of computer node 610 through the intranode interconnect 618 of computer node 610. In other embodiments, the memory devices of the computer nodes (e.g., memory devices 618 and 628 of computer nodes 610 and 620, respectively) may be subdivided into further domains either physically or virtually via an operating system of the computer node.

Referring to FIGS. 9 and 10, in some embodiments, a parallelism scheme such as the parallelism scheme 550 may be executed on a computer architecture such as the computer architecture 600. In some embodiments, a given MPI rank (e.g., MPI ranks 571-574) may access all the CPU cores of a computer node, all of the CPU cores of a given socket of a computer node, or to a subset of the CPUs of a given socket of a computer node. As an example, in some embodiments, MPI rank 571, when executed on computer architecture 600, may access each of the CPU cores 614 of computer node 610 or each of the CPU cores 624 of computer node 610. In other embodiments, MPI rank 571 may only access each CPU core 614 of one of the sockets 612 of computer node 610 or each CPU core 624 of one of the sockets 622 of computer node 620. In still other embodiments, MPI rank 571 may access only a subset and not the entirety of the CPU cores 614 of one of the sockets 612 of computer node 610 or a subset of the CPU cores 624 of one of the sockets 622 of computer node 620.

The allocation of CPU cores to a given MPI rank may be depend on how many MPI ranks are instantiated and the number of computer nodes available. The ratio of MPI ranks to number of computer nodes (e.g., the ratio of MPI ranks 571-574 to computer nodes 610 and 620 in this example) may impact performance depending on the specific computer architecture. However, in at least some embodiments, the ratio of MPI ranks instantiated on a given computer architecture to the number of computer nodes of the computer architecture is maintained between 1 and 8 to optimize the computing efficiency of the computer architecture.

In this exemplary embodiment, each MPI rank 571-574 of parallelism scheme 550 implements or executes a separate instance of an advection-diffusion solver 576 (e.g., four separate instances of advection-diffusion solver 576 are implemented by MPI ranks 571-574 in this example) in parallel on the corresponding chunk 554-557 of digital image 552 assigned to the respective MPI rank 571-574. In some embodiments, advection-diffusion solver 576 comprises one or more LB solvers. In this manner, MPI ranks 571-574 may concurrently and in parallel model advection and diffusion within at least some of the voxels 553 of the corresponding chunk 554-557 assigned to the given MPI rank 571-574.

As described above, each MPI rank may access several different CPU cores of a single socket of a computer node, or over multiple sockets of a computer node. For example, when executing the advection-diffusion solver 576, each MPI rank 571-574 may use each CPU core available to the MPI rank 571-574 to process the data. This is a multi-threading approach where the memory (e.g., memory devices 616, 626) is shared among all the CPU cores of computer nodes 610, 620. For instance, a given MPI rank 571-574 can operate on the entire chunk of data without needing to move memory from one non-uniform memory access (NUMA) node to another NUMA node. In addition, the calculations associated with halo regions 558-560 are exchanged between the MPI ranks 571-574 to permit continuous flow across the boundaries defined by chunks 554-557.

In addition to executing advection-diffusion solver 576 in parallel on the chunks 554-557 of digital image 552, in this exemplary embodiment of parallelism scheme 550, a geochemistry solver 590 (e.g., comprising the PHREEQC geochemical modeling software) is implemented or executed concurrently in parallel for each MPI rank 571-574 on the chunk 554-557 of digital image 552 assigned to the given MPI rank 571-574. In this manner, geochemical changes within at least some of the voxels 553 (e.g., the fluid and interface voxels) of each chunk 554-557 may be modeled in parallel using geochemical solver 590.

In this exemplary embodiment, a separate instance of the geochemical solver 590 is created for each CPU core available to the given MPI rank 571-574 (e.g., N instances of the geochemical solver 590 are created for the N CPU cores assigned to MPI rank 571). This one-to-one relationship between the instances of geochemical solver 590 created and the number of CPU cores available may be utilized in instances when a single instance of the geochemical solver 590 is limited to using only one CPU core at a given time due to the configuration of the geochemical solver 590, For example, a single instance of the PHREEQC geochemical modeling software (which may comprise geochemical solver 590) may be configured to use only one CPU core at a given time.

Thus, in this exemplary embodiment, multiple instances of the program may be loaded for a given MPI rank. Particularly, a first plurality of instances 591 of the geochemical solver 590 are implemented by the plurality of CPU cores 581 assigned to MPI rank 571, and a second plurality of instances 592 of the geochemical solver 590 are implemented by the plurality of CPU cores 582 assigned to MPI rank 572. In addition, a third plurality of instances 593 of the geochemical solver 590 are implemented by the plurality of CPU cores 583 assigned to MPI rank 573, and a fourth plurality of instances 594 of the geochemical solver 590 are implemented by the plurality of CPU cores 584 assigned to MPI rank 574. This process may provide better computational efficiency than letting a single instance of geochemical solver 590 access multiple CPU cores. However, in other embodiments, each instance of geochemical solver 590 may access a plurality of CPU cores. For instance, in an alternative embodiment, a single instance of geochemical solver 590 may be created for MPI rank 571 while a second instance of geochemical solver 590 is created for MPI rank 572 and so on and so forth.

In this exemplary embodiment, during the geochemistry calculation performed using the created instances 591-594 of geochemical solver 590, the interface voxels are divided, for example, roughly equally into the number of instances of the geochemical solver 590, and each separate instance of the geochemical solver 590 processes the chemical reactions and provides the results obtained therefrom to the advection-diffusion solver 576. For instance, instances 591 of geochemical solver 590 executed using CPU cores 581 assigned to MPI rank 571 provide their results to the instance of advection-diffusion solver 576 implemented by MPI rank 571 and so on and so forth. This process may be performed by dividing the data up evenly at the start of the initiation of the geochemistry solver 590. Alternatively, a queue of tasks may be created, where each instance of the of the geochemical solver 590 fetches the next task from the queue (e.g., using an asynchronous OpenMP tasking model), processes the geochemistry, provides result to the advection-diffusion solver 576, and returns to the queue to fetch the next item in the queue until the queue is empty.

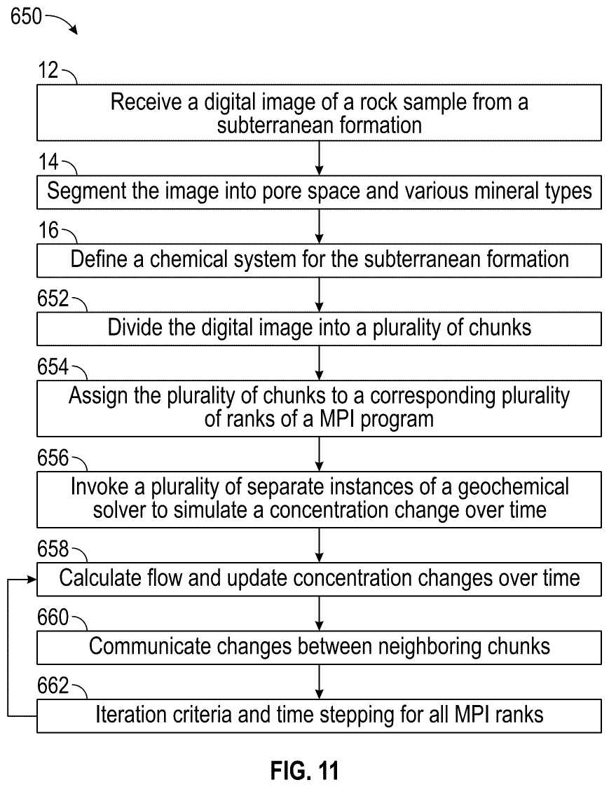

Referring now to FIG. 11, a flowchart of an embodiment of method 650 for geochemically modeling porous media such as a subterranean formation is shown. In some embodiments, method 650 may be implemented to simulate reactive transported in the subterranean formation in accordance with examples described herein. Particularly, in some embodiments, method 650 describes an example of a parallelism scheme which may be used to optimize reactive transport simulations within porous media as shown in FIG. 9. In some embodiments, at least some of the operations of method 650 may be performed by, with, or using instructions stored in a computer readable medium, and executed by one or more CPU cores of a computer system or device (or multiple computer devices). As an example, the computer device (or devices) may comprise or be similar to the example computer system 400 shown in FIG. 8 having a computer architecture similar to the example shown in FIG. 10. In addition, and as further discussed below, method 650 may incorporate at least some of the features or steps of method 10 described above and shown in FIG. 1.

The method 650 begins at block 12, with receiving a digital image (e.g., a 3D digital image) of a rock from a subterranean formation. The digital image may comprise a digital image generated from a natural rock sample obtained from a subterranean formation, or a synthetic rock sample. Method 650 continues at block 14 by segmenting the digital image of the rock sample into pore space and solid phase space. At block 16, method 650 includes defining a chemical system for the subterranean formation.

Method 650 continues at block 652 by dividing the image into a plurality of separate chunks. In some embodiments, each of the plurality of chunks comprises a separate portion of the plurality of voxels of the image. In other words, at least some of the voxels associated with a given chunk are unique to that chunk and not assigned to other chunks of the image. As an example, in some embodiments, block 652 includes dividing image 552 shown in FIG. 9 into the plurality of separate chunks 554-557, each chunk 554-557 including a plurality of separate or unique voxels 553 of the image 552.

As described above, the plurality of voxels (e.g., the voxels 553 of digital image 552 shown in FIG. 9) defining the digital image are distributed between separate chunks (e.g., chunks 554-557) whereby each chunk comprises a separate portion of the plurality of voxels of the digital image. In this exemplary embodiment, each chunk comprises a similar number of voxels (e.g., chunks 554-557 may each comprise a comparable number of voxels 553); however, in other embodiments, the number of voxels of each chunk may vary (e.g., for the purposes of load balancing). Additionally, at least in some embodiments, the plurality of chunks assigned to a rank may change (e.g., be redistributed) throughout the application of method 650 to ensure load balancing is maintained throughout the application. Additionally, in some embodiments, one or more of the chunks contains solid voxels, fluid voxels, and/or interface voxels. In some embodiments, block 652 additionally includes determining the desired volume of chunk relative to the total volume of the image. In other words, it may determine how many chunks should the image be divided between which may be based, at least in some embodiments, on the computer architecture available for implementing method 650.

Method 650 continues in block 654 with assigning the plurality of chunks of the image to a corresponding plurality of ranks of an MPI (e.g., MPI ranks) program. As an example, in some embodiments, block 654 includes assigning the chunks 554-557 of the image 552 shown in FIG. 9 to a corresponding plurality of MPI ranks 571-574 of the MPI program 570. In this manner, each MPI rank is assigned a unique chunk of the image (e.g., chunk 554 MPI rank 571 is assigned chunk 554, MPI rank 572 is assigned chunk 555, etc.).

The MPI ranks are implemented using a computer architecture such as, for example, the computer architecture 600 shown in FIG. 10. In some embodiments, block 654 includes dividing the MPI ranks evenly among one or more computer nodes or assigning each MPI rank to a different computer node (e.g., a different computer node 610 or 620 shown in FIG. 10) having a plurality of CPU cores (e.g., CPU cores 614 for computer node 610, and CPU cores 624 for computer node 620 as shown in FIG. 10)

In some embodiments, block 654 includes dividing the number of CPU cores of each computer node among the different MPI ranks. In this exemplary embodiment, each MPI rank is associated with a set of unique CPU cores, with each MPI rank loading a unique problem chunk into memory. For example, the MPI rank 571 when implemented using the computer architecture 600 shown in FIG. 10 may be assigned computer node 610 of architecture 600. In addition, in some embodiments, MPI rank 571 may be assigned each CPU core 614 of both sockets 612 of computer node 610. In other embodiments, MPI rank 571 may be assigned each CPU core 614 of only one of the sockets 612 of computer node 610. In still other embodiments, MPI rank 571 may be assigned some but not all of the CPU cores 614 of only one of the sockets 612 of computer node 610.

In certain embodiments, the available CPU cores are divided evenly between the different MPI ranks. For example, MPI rank 571 may be assigned each CPU core 614 of one socket 612 of computer node 610, MPI rank 572 may be assigned each CPU core 614 of the other socket 612 of computer node 610, MPI rank 573 may be assigned each CPU core 624 of one socket 622 of computer node 620, and MPI rank 574 may be assigned each CPU core 624 of the other socket 622 of computer node 620. However, in other embodiments, the number of CPU cores may be assigned unevenly between the different MPI ranks so as to maximize computational efficiency.

Method 650 continues in block 656 with invoking a plurality of separate instances of a geochemical solver to simulate concentration changes in the digital image over time. In some embodiments, block 656 comprises simulating solute concentration changes over time of the fluid phase of the image. In some embodiments, block 656 comprises implementing one or more advection-diffusion solvers for each MPI rank so as to simulate advection and diffusion of each solute in the fluid through the chunk to which the given MPI rank is assigned. For example, in some embodiments, block 656 comprises MPI ranks 571-574 shown in FIG. 9 each executing a separate instance (or instances) of the advection-diffusion solver 576 (e.g., a LB solver) to simulate advection and diffusion through the corresponding chunks 554-557 of the image 552.

In addition, in some embodiments, block 656 comprises executing for each MPI rank an instance of a geochemical solver (e.g., the PHREEQC geochemical modeling software) to simulate the chemical activity (e.g., chemical reactions) occurring in each corresponding chunk to which the MPI ranks are assigned. In certain embodiments, block 656 comprises executing a separate instance of the geochemical solver for each CPU core assigned to a given MPI rank. In this manner, the geochemical reaction simulations may be performed as a set of parallel independent calculations (e.g., performed simultaneously) across each CPU core assigned to the different MPI ranks. For instance, a first instance of the geochemical solver 590 may be executed by a first CPU core assigned to MPI rank 571, a second instance of the geochemical solver 590 may be executed by the second CPU core assigned to MPI rank 571, and so on and so forth. Executing a separate instance of the geochemical solver for each CPU core assigned to the different MPI ranks may be particularly beneficial in applications in which a single-core geochemical solver (e.g., a geochemical solver that cannot be executed as a single instance across multiple CPU cores) is used.

In some embodiments, fluid transport components of the digital image are computed within a multi-threaded, shared memory across all of the separate CPU cores of the respective computer architecture. In this way, data movement and communication overhead are minimized between the separate CPU cores, maximizing the computation efficiency of the process. In some embodiments, a MPI is used for scaling across the computing system comprising the plurality of separate CPU cores as described above. Method 650 continues at block 658 by calculating flow and updating concentration changes over time. In some embodiments, block 658 includes modeling the concentration changes in the fluid and solid phase via reactive transport simulation using the parallelism scheme disclosed herein. In some embodiments, block 658 includes implementing blocks 18 through 26 of the method 10 of FIG. 1. Thus, in some embodiments, block 658 includes determining (in parallel by different CPU cores of a given computer architecture) the geochemical changes of the fluid concentrations and the solid phase mineral concentrations and the flow of the fluid concentrations via advection and diffusion as described further above with respect to block 22 of method 10. Additionally, in some embodiments, block 658 includes changes to the categorization of the voxels of the digital image as described further above with respect to block 24 of method 10. Additionally, in some embodiments, block 658 includes providing the model output as described further above with respect to block 26 of method 10.

Method 650 continues at block 660 by communicating changes between neighboring chunks. In some embodiments, block 660 includes communicating the results of the calculations in block 658 for each chunk across the MPI boundaries to permit continuous flow across the boundaries (e.g., between neighboring chunks assigned to separate MPI ranks). Method 650 continues at block 662 with the iteration criteria for all the MPI ranks. In some embodiments, block 662 includes implementing blocks 28 through 32 of the method 10 in FIG. 1. Thus, in some embodiments, blocks 662 includes an evaluation of if the time stamp is greater than or equal to the final time stamp as described further above with respect to block 28 of method 10. Additionally, in some embodiments, block 658 includes evaluating the global porosity change across all the chunks as described further above in block 32 of the method 10.

The discussion above is directed to various exemplary embodiments. However, one of ordinary skill in the art will understand that the examples disclosed herein have broad application, and that the discussion of any embodiment is meant only to be exemplary of that embodiment, and not intended to suggest that the scope of the disclosure, including the claims, is limited to that embodiment.

The drawing figures are not necessarily to scale. Certain features and components herein may be shown exaggerated in scale or in somewhat schematic form and some details of conventional elements may not be shown in interest of clarity and conciseness.

In the discussion above and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. In addition, when used herein (including in the claims), the words “about,” “generally,” “substantially,” “approximately,” and the like mean within a range of plus or minus 10%.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A computer-implemented method for modeling reactive transport within a subterranean formation, the method comprising:
(a) receiving a digital image of rock from the subterranean formation;
(b) defining a chemical system for the digital image;
(c) segmenting the digital image into a plurality of voxels including a plurality of solid voxels each associated with a solid phase mineral of the subterranean formation, a plurality of fluid voxels each associated with a fluid of the subterranean formation, and a plurality of interface voxels each associated with both the solid phase mineral and the fluid of the subterranean formation;
(d) simulating a concentration change over time of the solid phase mineral of each of the plurality of interface voxels due to simulated chemical activity in the digital image; and
(e) determining an updated concentration of the solid phase mineral for each of the plurality of interface voxels of the digital image based on the simulated concentration change.

2. The method of claim 1, further comprising:
(f) converting at least one of the plurality of interface voxels into either a solid voxel or a fluid voxel based on the updated concentration of the solid phase mineral for each of the plurality of interface voxels of the digital image.

3. The method of claim 2, wherein (f) comprises updating a change in pore space of the digital image based on the simulated concentration change and a geometry of an interface of the digital image defined by the spatial configuration of the plurality of interface voxels.

4. The method of claim 1, further comprising:
(f) converting at least one of the plurality of interface voxels into a solid voxel in response to the updated concentration of the solid phase mineral of at least one of the plurality of interface voxels equaling a corresponding concentration of the solid phase mineral at a full density or molar volume of the solid phase mineral, wherein the concentration of the solid phase mineral corresponds to the number of mols of the solid phase mineral present in the interface voxel.

5. The method of claim 4, wherein the at least one of the plurality of solid voxels is converted into the at least one of the plurality of interface voxels, and is located adjacent the at least one of the plurality of interface voxels having the updated concentration of the solid phase mineral equaling the density of the solid phase mineral.

6. The method of claim 1, further comprising:
(f) converting at least one of the plurality of interface voxels into a fluid voxel in response to the updated concentration of the solid phase mineral of at least one of the plurality of interface voxels equaling zero.

7. The method of claim 6, wherein the at least one of the plurality of fluid voxels is located adjacent the at least one of the plurality of interface voxels having the updated concentration of the solid phase mineral equal to zero.

8. The method of claim 1, wherein (d) comprises:
(d1) independently performing a geochemical calculation between the solid phase mineral and the fluid for each of the plurality of interface voxels;
(d2) simulating the concentration change over time of the solid phase mineral of each of the plurality of interface voxels based on a simulated change in a concentration of the fluid of the interface voxel and a predefined stochiometric relationship between the solid phase mineral and a solute component of the fluid; and
(d3) determining a change in temperature associated with the concentration change over time of the solid phase mineral.

9. A system for modeling reactive transport within a subterranean formation, the system comprising:
a processor; and
a memory coupled to the processor, wherein machine-readable instructions are stored in the memory, and wherein the machine-readable instructions, when executed on the processor, configure the processor to:
(a) receiving a digital image of rock from the subterranean formation;
(b) defining a chemical system for the digital image;
(c) segmenting the digital image into a plurality of voxels including a plurality of solid voxels each associated with a solid phase mineral of the subterranean formation, a plurality of fluid voxels each associated with a fluid of the subterranean formation, and a plurality of interface voxels each associated with both the solid phase mineral and the fluid of the subterranean formation;
(d) simulating a concentration change over time of the solid phase mineral of each of the plurality of interface voxels due to simulated chemical activity in the digital image; and
(e) determining an updated concentration of the solid phase mineral for each of the plurality of interface voxels of the digital image based on the simulated concentration change.

10. The system of claim 9, wherein the machine-readable instructions, when executed on the processor, configure the processor to:
(f) converting at least one of the plurality of interface voxels into a solid voxel in response to the updated concentration of the solid phase mineral of at least one of the plurality of interface voxels equaling a corresponding concentration of the solid phase mineral at a full density or molar volume of the solid phase mineral, wherein the concentration of the solid phase mineral corresponds to the number of mols of the solid phase mineral present in the interface voxel.

11. The system of claim 9, wherein the digital image of rock is a three-dimensional (3D) image.

12. A computer-implemented method for modeling reactive transport within a subterranean formation, the method comprising:
(a) receiving a digital image of rock from the subterranean formation;
(b) defining a chemical system for the digital image;
(c) segmenting the digital image into a plurality of voxels at least some of which are associated with a solid phase mineral of the subterranean formation and at least some of which are associated with a fluid of the subterranean formation;
(d) dividing the digital image into a plurality of chunks, each chunk comprising a separate portion of the plurality of voxels of the digital image;
(e) assigning the plurality of chunks to a corresponding plurality of ranks of a message passing interface (MPI) program such that each rank is associated with a unique chunk of the digital image;
(f) invoking, for each of the plurality of MPI ranks, a plurality of separate instances of a geochemical solver to simulate, in parallel, a concentration change over time of at least one of the solid phase mineral and the fluid of the digital image due to simulated transport and chemical activity in the digital image; and
(g) determining an updated concentration of the at least one of the solid phase mineral and the fluid of the digital image based on the simulated concentration change.

13. The method of claim 12, further comprising:
(h) segmenting the digital image is segmented into a plurality of voxels which are divided between the plurality of chunks of the digital image whereby each chunk is associated with multiple distinct voxels of the plurality of voxels.

14. The method of claim 12, further comprising:
(h) assigning a unique computer node of a computer architecture to each of the plurality of MPI ranks; and
wherein (f) comprises executing at least one of an advection-diffusion solver and the geochemical solver on the computer nodes assigned to the plurality of MPI ranks.

15. The method of claim 12, wherein each of the plurality of geochemical solver instances is assigned to a unique central processing unit (CPU) core of a computer architecture.

16. The method of claim 12, further comprising:
(h) assigning a unique set of central processing unit (CPU) cores of one or more computer nodes of a computer architecture to each of the plurality of MPI ranks; and
wherein (f) comprises executing at least one of an advection-diffusion solver and the geochemical solver on the CPU cores assigned to the plurality of MPI ranks.

17. The method of claim 16, wherein (f) comprises executing a separate instance of the at least one of the advection-diffusion solver and the geochemical solver for each of the CPU cores assigned to the plurality of MPI ranks.

18. The method of claim 12, further comprising:
(h) assigning a unique socket of one or more computer nodes of a computer architecture to each of the plurality of MPI ranks; and
wherein (f) comprises executing at least one of an advection-diffusion solver and the geochemical solver on the sockets assigned to the plurality of MPI ranks.

19. The method of claim 12, further comprising:
(h) communicating between the plurality of MPI ranks information pertaining to the digital image across an internode interconnect connected between a plurality of computer nodes of a computer architecture assigned to the plurality of MPI ranks.

20. The method of claim 12, further comprising:
(h) communicating between the plurality of MPI ranks information pertaining to the digital image across an intranode interconnect connected between a plurality of sockets of a computer node assigned to the plurality of MPI ranks.
